(19) United States
(12) Patent Application Publication
(10) Pub. No.: US 2026/0188487 A1
(43) Pub. Date: Jul. 2, 2026

(54) APPARATUS AND METHOD FOR PREDICTING RECURRENCE OF ATRIAL FIBRILLATION

(71) Applicant: UIF (University Industry Foundation), Yonsei University, Seoul (KR)

(72) Inventors: Hui Nam PAK, Seoul (KR); Oh Seok KWON, Seoul (KR)

(73) Assignee: UIF (University Industry Foundation), Yonsei University, Seoul (KR)

(21) Appl. No.: 18/856,380

(22) PCT Filed: Mar. 27, 2023
(86) PCT No.: PCT/KR2023/004023

§ 371 Date: Oct. 11, 2024

(30) Foreign Application Priority Data
Apr. 14, 2022 (KR) 10-2022-0046544

(51) Int. Cl. G16H 50/20; G06T 5/20; G06T 5/70; G06T 7/00; G06T 7/11; G06T 7/62

(52) U.S. Cl. CPC G16H 50/20; G06T 5/20; G06T 5/70; G06T 7/0012; G06T 7/11; G06T 7/62; G06T 2207/10081; G06T 2207/20024; G06T 2207/20076; G06T 2207/20081; G06T 2207/20084; G06T 2207/20172

(57) ABSTRACT

A method for predicting a recurrence of atrial fibrillation by an apparatus comprising a processor and a memory, according to an embodiment of the present invention, comprises the steps of: (a) generating target data including any one or more of geometric topological data, anatomical data, and electrophysiological data of the atrium of a patient; (b) preprocessing the generated target data; and (c) inputting the preprocessed target data into a prediction model to calculate and output a prediction value of a recurrence of atrial fibrillation for the patient and to learn the prediction value, wherein the target data is defined as a plurality of vertices and edges and is data having a basic structure of a three-dimensional polygon mesh including a plurality of triangular faces, which are minimum units of faces including the vertices and edges.

### TECHNICAL FIELD

The present invention relates to an apparatus and method for predicting recurrence of atrial fibrillation. More specifically, the present invention relates to an apparatus and method for predicting recurrence of atrial fibrillation by inputting three-dimensional mapping data into an artificial intelligence model.

### BACKGROUND ART

Arrhythmia refers to a symptom of abnormally fast, slow, or irregular heartbeats as regular contraction is not continued due to poor electrical stimulation or improper transmission of impulses in the heart, and the main cause thereof is atrial fibrillation, and in a severe case, it may lead to a sudden death or stroke.

Meanwhile, there is a problem in that the diagnosis is relatively late as 40 to 80% of atrial fibrillation occur without a symptom. Conventionally, diagnosis is performed by monitoring electrical activities of the heart through an electrocardiogram, but since the change in the electrical activities appearing on the electrocardiogram is a sign that it is highly possible to be developed into chronic atrial fibrillation, it needs to provide an appropriate treatment method by diagnosing atrial fibrillation in the early stage to prevent development into chronic atrial fibrillation or furthermore into arrhythmia.

Although some techniques developed recently therefor predict possibility of occurrence of the atrial fibrillation from normal rhythm electrocardiograms, the techniques require a very large amount of information and are highly vulnerable to noise due to the nature of recording electrical activities, and this may result in misdiagnosis.

The present invention relates to a new and innovative technique that can accurately and rapidly predict recurrence of atrial fibrillation with only minimal information by reflecting the problems of conventional techniques.

### DISCLOSURE OF INVENTION

### Technical Problem

Therefore, the present invention has been made in view of the above problems, and it is an object of the present invention to provide an apparatus and method of predicting recurrence of atrial fibrillation, which can rapidly and accurately predict recurrence of atrial fibrillation with only minimal information.

Another object of the present invention is to provide an apparatus and method of predicting recurrence of atrial fibrillation, which can predict recurrence of atrial fibrillation by utilizing information previously possessed by the patient, without the need of acquiring new information through invasive tests that have not been performed for patients previously to predict recurrence of atrial fibrillation.

The technical problems of the present invention are not limited to the technical problems mentioned above, and unmentioned other technical problems will be clearly understood by those skilled in the art from the following description.

### Technical Solution

To accomplish the above objects, according to one aspect of the present invention, there is provided a method of predicting recurrence of atrial fibrillation through an apparatus including a processor and a memory, the method comprising the steps of: (a) generating target data including any one or more among geometric topological data, anatomical data, and electrophysiological data of a patient's atrium; (b) performing preprocessing on the generated target data; and (c) inputting the preprocessed target data into a prediction model, calculating and outputting a prediction value of recurrence of atrial fibrillation of the patient, and learning the prediction value, wherein the target data is defined by a plurality of vertices and edges and adopts a three-dimensional polygon mesh including a plurality of triangular faces, i.e., minimum units of faces configured of vertices and edges, as a basic structure.

According to an embodiment, the geometric topological data of the patient's atrium may be data generated as a three-dimensional polygon mesh by applying a marching cubes algorithm while structures adjacent to the heart are excluded from a computed tomography (CT) image of the patient's heart and only segmented shapes of the atrium are extracted.

According to an embodiment, the anatomical data of the patient's atrium may be data that maps information on atrial wall thickness at all points included in the patient's atrium expressed as a three-dimensional polygon mesh, which is the geometric topological data of the patient's atrium.

According to an embodiment, the electrophysiological data of the patient's atrium may include first electrophysiological data that maps information on local activation time at all points included in the patient's atrium expressed as a three-dimensional polygon mesh, which is the geometric topological data of the patient's atrium.

According to an embodiment, the electrophysiological data of the patient's atrium may be generated using data that maps voltage information at all points included in the patient's atrium expressed as a three-dimensional polygon mesh, which is the geometric topological data of the patient's atrium, as well as the first electrophysiological data, and may include second electrophysiological data that maps information on fibrosis at all points included in the patient's atrium expressed as a three-dimensional polygon mesh, which is the geometric topological data of the patient's atrium.

According to an embodiment, step (b) may include the steps of: (b-1) performing mesh smoothing, which is first preprocessing, on the generated target data; (b-2) performing mesh simplification, which is second preprocessing, on the target data on which the mesh smoothing has been performed; and (b-3) performing generation of N three-dimensional polygon-derived meshes (N is a positive integer), which is third preprocessing, on the target data on which the mesh simplification has been performed, and merging the generated mesh with the target data.

According to an embodiment, the mesh smoothing, which is first preprocessing performed at step (b-1), may be performed by applying a Laplacian smoothing algorithm, and an iterative operation for the Laplacian Smoothing may be performed three times.

According to an embodiment, the mesh simplification, which is second preprocessing, performed at step (b-2) may include the steps of: (b-2-1) selecting uniform vertices among the plurality of vertices by applying a Poisson disk sampling algorithm; and (b-2-2) simplifying the selected uniform vertices by applying an edge collapse algorithm, wherein at step (b-2-2), the mesh may be configured of uniform vertices based on the selected uniform vertices while preserving a mesh surface by applying a re-tiling algorithm.

According to an embodiment, the mesh simplification, which is second preprocessing, performed at step (b-2) may be performed under a control condition in which a reference geometric shape (Hausdorff distance) is less than 2 and a structural similarity (SSIM) is less than 0.7.

According to an embodiment, the target data on which the method has been performed as far as step (b-2) may be a three-dimensional polygon mesh structure including 2,048 vertices, 6,144 edges, and 4,096 faces.

According to an embodiment, in generation of derived meshes at step (b-3), which is third preprocessing, N may be 5, and the three-dimensional polygon-derived meshes may be three-dimensional polygon meshes having a shape the same as that of the target data on which the mesh simplification has been performed, and may be meshes having edges to which the vertices are connected are different.

According to an embodiment, step (c) may include the steps of: (c-1) calculating edge-based features by inputting the preprocessed target data into the prediction model; (c-2) calculating a first output value by inputting the extracted edge-based features into the prediction model and applying mesh convolution and mesh pooling; and (c-3) calculating and outputting the prediction value of recurrence of atrial fibrillation of the patient, which is a second output value, by applying soft voting to the calculated first output value.

According to an embodiment, when the target data includes geometric topological data, step (c-1) may include (c-1-1) a step of inputting the preprocessed target data into the prediction model and calculating a diameter at all edges, which is a first edge-based feature, wherein the diameter at all the edges may be calculated by estimating collision points from each edge to an opposite surface through ray casting.

According to an embodiment, when the target data includes any one or more among anatomical data and electrophysiological data, step (c-1) may include (c-1-2) a step of inputting the preprocessed target data into the prediction model and calculating mapping information at all edges, which is a second edge-based feature, wherein the mapping information at all edges may be calculated by estimating a center of gravity of two triangles including any one edge for which mapping information is to be calculated, estimating a distance from the center of gravity to a center point of any one edge, and applying a linear interpolation method.

According to another aspect of the present invention, there is provided an apparatus for predicting recurrence of atrial fibrillation, the apparatus comprising: one or more processors; a network interface; a memory that loads a computer program executed by the processor; and a storage that stores a large amount of network data and the computer program, wherein the computer program executes, by the one or more processors, (A) an operation of generating target data including any one or more among geometric topological data, anatomical data, and electrophysiological data of a patient's atrium; (B) an operation of performing preprocessing on the generated target data; and (C) an operation of inputting the preprocessed target data into a prediction model, calculating and outputting a prediction value of recurrence of atrial fibrillation of the patient, and learning the prediction value, wherein the target data is defined by a plurality of vertices and edges and adopts a three-dimensional polygon mesh including a plurality of triangular faces, i.e., minimum units of faces configured of vertices and edges, as a basic structure.

According to another aspect of the present invention, there is provided a computer program stored in a medium, the program comprising, in combination with a computing device, the steps of: (AA) generating target data including any one or more among geometric topological data, anatomical data, and electrophysiological data of a patient's atrium; (BB) performing preprocessing on the generated target data; and (CC) inputting the preprocessed target data into a prediction model, calculating and outputting a prediction value of recurrence of atrial fibrillation of the patient, and learning the prediction value, wherein the target data is defined by a plurality of vertices and edges and adopts a three-dimensional polygon mesh including a plurality of triangular faces, i.e., minimum units of faces configured of vertices and edges, as a basic structure.

### Advantageous Effects

According to the present invention as described above, there is an effect of rapidly predicting recurrence of atrial fibrillation with high accuracy using only computed tomography images, information on atrial wall thickness, information on local activation time, and information on fibrosis, which are the minimum level of information on the patient's heart.

In addition, since the information described above is information generally possessed by a patient of atrial fibrillation and is also information definitively generated by present invention through an operation, a new invasive test, which is burdensome to the patient's body, does not need to be performed to acquire new information, and accordingly, there is an effect of minimizing physical and economic burdens of the patient.

In addition, since a prediction value of recurrence of atrial fibrillation is calculated and automatically output by simply generating and inputting target data into a prediction model, and the prediction value is continuously learned, there is an effect of improving accuracy of predicting recurrence of atrial fibrillation as the present invention is repeatedly used.

The effects of the present invention are not limited to the effects mentioned above, and unmentioned other effects will be clearly understood by those skilled in the art from the following description.

### BRIEF DESCRIPTION OF THE DRAWINGS

FIG. 1 is a view showing the overall configuration included in an apparatus for predicting recurrence of atrial fibrillation according to a first embodiment of the present invention.

FIG. 2 is a flowchart illustrating representative steps of a method of predicting recurrence of atrial fibrillation according to a second embodiment of the present invention.

FIG. 3 is a view exemplarily showing three vertices, three edges connecting the vertices, and one triangular face configured of the vertices and edges.

FIG. 4 is a view exemplarily showing a process of generating geometric topological data of a patient's atrium.

FIG. 5 is a view exemplarily showing geometric topological data of a patient's atrium, which is finally generated by applying a marching cubes algorithm.

FIG. 6 is a view exemplarily showing anatomical data of a patient's atrium.

FIG. 7 is a view exemplarily showing first electrophysiological data included in electrophysiological data of a patient's atrium.

FIG. 8 is a view exemplarily showing data that maps voltage information at all points included in a patient's atrium.

FIG. 9 is a view exemplarily showing second electrophysiological data included in electrophysiological data of a patient's atrium.

FIG. 10 is a flowchart specifically illustrating step S220 that performs preprocessing on target data in a method of predicting recurrence of atrial fibrillation according to a second embodiment of the present invention.

FIG. 11 is a view exemplarily showing a view of edge simplification according to application of an edge collapse algorithm.

FIG. 12 is a view exemplarily showing the results of performing the method of predicting recurrence of atrial fibrillation as far as mesh simplification, which is the second preprocessing of step S220-2, on the data that maps voltage information at all points included in the patient's atrium of three patients A, B, and C.

FIG. 13 is a view exemplarily showing a case where topologies are different although the shapes are the same.

FIG. 14 is a view exemplarily showing anatomical data of a patient's atrium, which is the target data, and a view of five three-dimensional polygon-derived meshes of the anatomical data.

FIG. 15 is a flowchart specifically illustrating step S230 of calculating and outputting a prediction value of recurrence of atrial fibrillation in a method of predicting recurrence of atrial fibrillation according to a second embodiment of the present invention.

FIG. 16 is a view exemplarily showing a view of calculating collision points from each edge to an opposite surface using a ray casting method.

FIG. 17 is a view exemplarily showing a view of calculating mapping information at all edges.

FIG. 18 is a view exemplarily showing a view of calculating a first output value by inputting target data into a prediction model, and calculating and outputting a prediction value of recurrence of atrial fibrillation of a patient, which is a second output value, by applying soft voting to the first output value.

FIG. 19 is a view exemplarily showing a single mesh neural network constituting a prediction model.

FIG. 20 is ROC curves showing the results of long-term observation of recurrence of atrial fibrillation by year recorded in the EMR based on data of patients with recurrence of atrial fibrillation after actual radiofrequency catheter ablation, in a method of predicting recurrence of atrial fibrillation according to a second embodiment of the present invention.

FIG. 21 is a table showing the results of long-term observation of recurrence of atrial fibrillation by year recorded in the EMR based on data of patients with recurrence of atrial fibrillation after actual radiofrequency catheter ablation, in a method of predicting recurrence of atrial fibrillation according to a second embodiment of the present invention.

### BEST MODE FOR CARRYING OUT THE INVENTION

Hereinafter, preferred embodiments of the present invention will be described in detail with reference to the accompanying drawings. The advantages and features of the present invention and the method for achieving them will become clear by referring to the embodiments described below in detail together with the accompanying drawings. However, the present invention is not limited to the embodiments disclosed below and will be implemented in various different forms. These embodiments are provided only to make the disclosure of the present invention complete and to fully inform those skilled in the art of the scope of the present invention, and the present invention is only defined by the scope of the claims. Like reference numerals refer to like elements throughout the specification.

Unless defined otherwise, all the terms (including technical and scientific terms) used in this specification may be used as meanings that can be commonly understood by those skilled in the art.

In addition, terms defined in commonly used dictionaries are not interpreted ideally or excessively unless clearly and specifically defined.

The terms used in this specification are to describe the embodiments and are not to limit the present invention. In this specification, singular forms also include plural forms unless specifically stated otherwise in the phrases.

The terms “comprises” and/or “comprising” used in this specification means that the mentioned components, steps, operations, and/or elements do not exclude the presence or addition of one or more other components, steps, operations and/or elements.

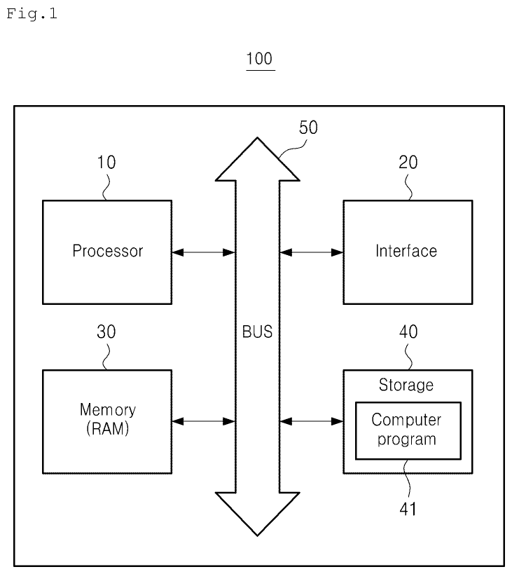

FIG. 1 is a view showing the overall configuration included in an apparatus 100 for predicting recurrence of atrial fibrillation according to a first embodiment of the present invention.

However, this is only a preferred embodiment for achieving the objects of the present invention, and some components may be added or deleted as needed, and other components may also perform the functions performed by any one component.

An apparatus 100 for predicting recurrence of atrial fibrillation according to a first embodiment of the present invention may include a processor 10, a network interface 20, a memory 30, a storage 40, and a data bus 50 connecting them.

The processor 10 controls the overall operation of each component. The processor 10 may be any one among a Central Processing Unit (CPU), a Micro Processor Unit (MPU), a Micro Controller Unit (MCU), and a processor of a type widely known in the art. In addition, the processor 10 may perform operations of at least one application or program to perform a method of predicting recurrence of atrial fibrillation according to a second embodiment of the present invention, and it is preferable to implement as an artificial intelligence processor.

The network interface 20 supports wired and wireless Internet communication of the apparatus 100 for predicting recurrence of atrial fibrillation according to a first embodiment of the present invention, and may also support other known communication methods. Accordingly, the network interface 20 may be configured to include a communication module according thereto.

The memory 30 may store various data, commands and/or information, and load one or more computer programs 41 from the storage 40 to perform the method of predicting recurrence of atrial fibrillation according to a second embodiment of the present invention. Although RAM is shown as a kind of the memory 30 in FIG. 1, it goes without saying that various storage media can be used as the memory 30.

The storage 40 may non-temporarily store one or more computer programs 41 and a large amount of network data 42. The storage 40 may be any one among a non-volatile memory such as read only memory (ROM), erasable programmable ROM (EPROM), electrically erasable programmable ROM (EEPROM), flash memory or the like, a hard disk, a detachable disk, and any type of computer-readable recording medium widely known in the art.

The computer program 41 may be loaded on the memory 30 and execute, by one or more processors 10, (A) an operation of generating target data including any one or more among geometric topological data, anatomical data, and electrophysiological data of a patient's atrium; (B) an operation of performing preprocessing on the generated target data; and (C) an operation of inputting the preprocessed target data into a prediction model, and calculating and outputting a prediction value of recurrence of atrial fibrillation of the patient. The target data is defined by a plurality of vertices and edges, and may be data that adopts a three-dimensional polygon mesh including a plurality of triangular faces, i.e., minimum units of faces configured of vertices and edges, as a basic structure.

The operations performed by the computer program 41 briefly mentioned so far may be regarded as a function of the computer program 41, and a more detailed description will be provided below in the description of the method of predicting recurrence of atrial fibrillation according to a second embodiment of the present invention.

The data bus 50 functions as a passage of commands and/or information between the processor 10, the network interface 20, the memory 30, and the storage 40 described above.

The apparatus 100 for predicting recurrence of atrial fibrillation according to a first embodiment of the present invention described above may be a physically independent electronic device, but it may be implemented as a function of a server (not shown) operated by a medical institution such as a hospital or the like since it may need to receive some information about the patient's atrium from the server. In this case, the server may be a tangible physical server or a virtual cloud server.

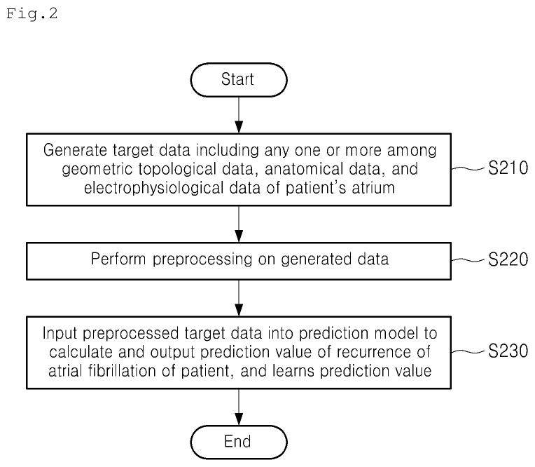

Hereinafter, a method of predicting recurrence of atrial fibrillation according to a second embodiment of the present invention will be described with reference to FIGS. 2 to 21.

FIG. 2 is a flowchart illustrating representative steps of a method of predicting recurrence of atrial fibrillation according to a second embodiment of the present invention.

This is only a preferred embodiment for achieving the objects of the present invention, and some steps may be added or deleted as needed, and furthermore, any one step may be included in another step.

Meanwhile, it is assumed that all steps are performed by the apparatus 100 for predicting recurrence of atrial fibrillation according to a first embodiment of the present invention, and hereinafter, it is simply named as an apparatus 100 including a processor and a memory.

First, the apparatus 100 including a processor and a memory generates target data including any one or more among geometric topological data, anatomical data, and electrophysiological data of a patient's atrium (S210).

Although it is sufficient that the target data generated herein only includes any one or more among geometric topological data, anatomical data, and electrophysiological data of a patient's atrium, it is desirable to include all of them as much as possible to prevent misdiagnosis and improve accuracy of predicting recurrence of atrial fibrillation.

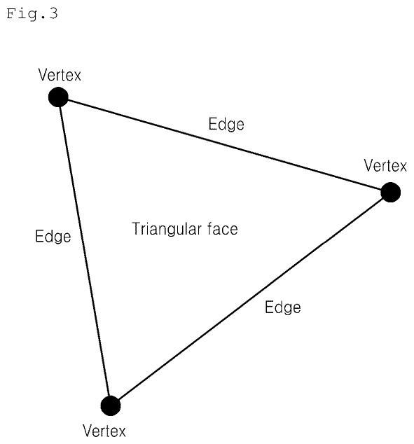

Meanwhile, the generated target data is data that is defined by a plurality of vertices and edges and adopts a three-dimensional polygon mesh including a plurality of triangular faces, i.e., minimum units of faces configured of vertices and edges, as a basic structure. FIG. 3 exemplarily shows three vertices, three edges connecting the vertices, and one triangular face configured of the vertices and edges. Since the target data is data of a patient's atrium, it may be data that adopts a three-dimensional polygon mesh structure of a patient's atrium including a plurality of triangular faces exemplarily shown in FIG. 3 as a basic structure. Hereinafter, geometric topological data, anatomical data, and electrophysiological data, which are detailed data included in the target data, will be described in order.

The geometric topological data of a patient's atrium is data generated as a three-dimensional polygon mesh by applying a marching cubes algorithm while structures adjacent to the heart are excluded from a computed tomography (CT) image of the patient's heart and only segmented shapes of the atrium are extracted.

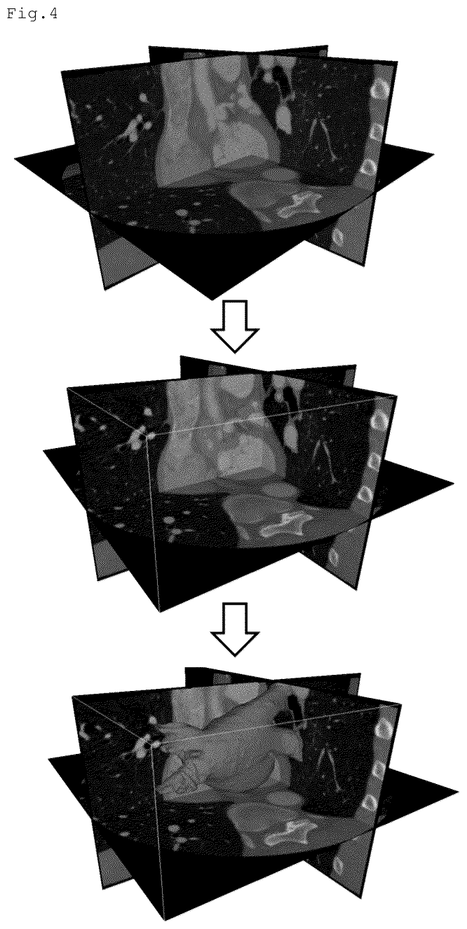

Since the geometric topological data like this means data of a three-dimensional polygon mesh as is, which is the basic structure of the target data, the basic structure of the target data may be the geometric topological data itself of the patient's atrium. The process of generating geometric topological data of the patient's atrium is exemplarily shown in FIG. 4, and describing the process in detail, an image is acquired by performing computed tomography while a contrast agent is injected into the blood vessel (drawing in the upper part of FIG. 4), and then only the shape of the atrium, excluding adjacent structures such as the lungs or the spine (drawing in the middle of FIG. 4), is extracted from the acquired image and segmented, and then a three-dimensional atrium model is generated (drawing in the lower part of FIG. 4), and the marching cubes algorithm is applied.

Meanwhile, the process of segmenting and extracting only the shape of the atrium and generating a three-dimensional atrial model may equally adopt a method of generating a three-dimensional left atrial model from a computed tomography image described in Patent Publication No. 10-2020-0095967A (Aug. 11, 2020), and since the marching cubes algorithm is also known, detailed description thereof will be omitted to avoid duplicate description.

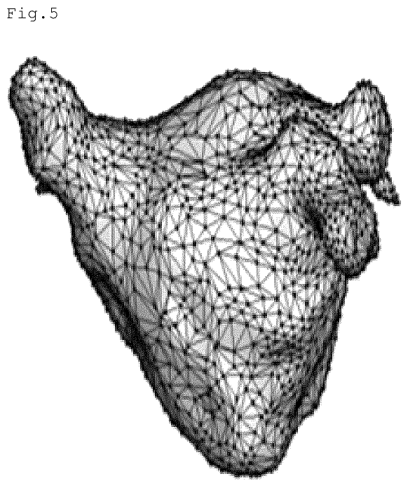

The geometric topological data of the patient's atrium finally generated by applying a marching cubes algorithm is exemplarily shown in FIG. 5, and since the shape of a plurality of vertices, edges connecting the vertices, and a triangular face configured of the vertices and edges is a shape that looks like a net, it is called a mesh, and each vertex has location information (x, y, z) of the Euclidean coordinate system.

The anatomical data of the patient's atrium is data that maps information on atrial wall thickness at all points included in the patient's atrium expressed as a three-dimensional polygon mesh, which is the geometric topological data of the patient's atrium.

Since the anatomical data like this is literally data that maps information on atrial wall thickness to the geometric topological data of the patient's atrium, it is data that can be generated after the geometric topological data of the patient's atrium is generated, and accordingly, the two data may be regarded as being in a dependent relation.

Meanwhile, all points included in the patient's atrium have a meaning different from the vertices included in the three-dimensional polygon mesh, which is the geometric topological data of the patient's atrium, and it is since that although the vertices included in the three-dimensional polygon mesh are the basic structural units of polygons and mean vertices where an edge exists between two vertices, all points included in the patient's atrium literally mean all points in the patient's atrium, and there are no edges or lines between the points, and generally, it is regarded that the number of all points included in the patient's atrium is larger than the number of vertices.

Since the process of mapping information on atrial wall thickness at all points included in the patient's atrium may adopt a method of estimating the thickness of the left atrial wall described in Patent Publication No. 10-2020-0095967A (Aug. 11, 2020) like generation of a three-dimensional atrial model as described above, detailed description thereof will be omitted to avoid duplicate description.

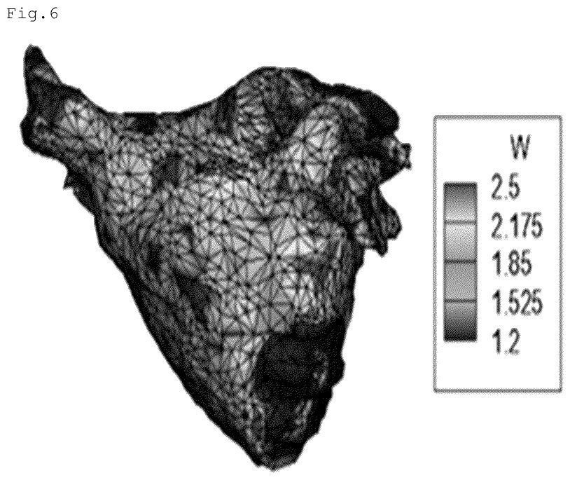

The anatomical data of a patient's atrium is exemplarily shown in FIG. 6, and since it is data that maps information on atrial wall thickness to a three-dimensional polygon mesh, which is the geometric topological data of the patient's atrium, it can be confirmed that it includes the geometric topological data shown in FIG. 5, more specifically, a plurality of vertices and edges, and a plurality of triangular faces configured of the vertices and edges, and since it can be confirmed that a predetermined color is shown on the surface of the atrium, each color represents the atrial wall thickness at all points included in the patient's atrium, and in this case, it can be said that information on the atrial wall thickness, as well as location information (x, y, z) of the Euclidean coordinate system, is mapped to each vertex included in the three-dimensional polygon mesh.

The electrophysiological data of the patient's atrium includes first electrophysiological data that maps information on local activation time at all points included in the patient's atrium expressed as a three-dimensional polygon mesh, which is the geometric topological data of the patient's atrium.

Since the first electrophysiological data like this is literally data that maps information on local activation time to the geometric topological data of the patient's atrium, it is data that can be generated after the geometric topological data of the patient's atrium is generated, and accordingly, the two data may be regarded as being in a dependent relation, like the anatomical data of the patient's atrium.

Meanwhile, since the process of mapping information on local activation time at all points included in the patient's atrium may adopt a method of generating (mapping) a potential activation map based on a patient's heart model described in Patent Registration No. 10-1964918B1 (Mar. 27, 2019) as it is, detailed description thereof will be omitted to avoid duplicate description.

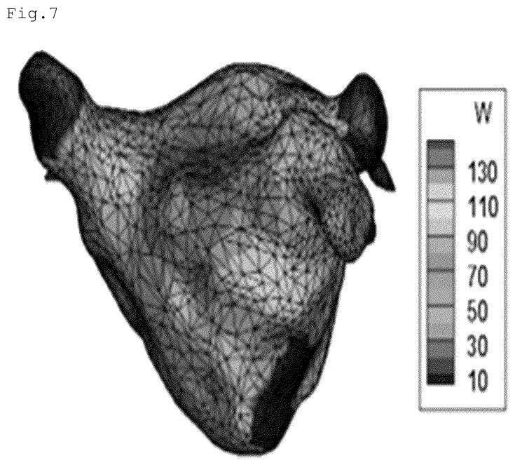

The first electrophysiological data included in the electrophysiological data of a patient's atrium is exemplarily shown in FIG. 7, and since it is data that maps information on atrial wall thickness to a three-dimensional polygon mesh, which is the geometric topological data of the patient's atrium, it can be confirmed that it includes the geometric topological data shown in FIG. 5, more specifically, a plurality of vertices and edges, and a plurality of triangular faces configured of the vertices and edges, and since it can be confirmed that a predetermined color is shown on the surface of the atrium, each color represents the local activation time at all points included in the patient's atrium, and in this case, it can be said that information on local activation time, as well as location information (x, y, z) of the Euclidean coordinate system, is mapped to each vertex included in the three-dimensional polygon mesh.

The electrophysiological data of the patient's atrium is generated using data that maps voltage information at all points included in the patient's atrium expressed as a three-dimensional polygon mesh, which is the geometric topological data of the patient's atrium, as well as the first electrophysiological data, and includes second electrophysiological data that maps information on fibrosis at all points included in the patient's atrium expressed as a three-dimensional polygon mesh, which is the geometric topological data of the patient's atrium.

Since the second electrophysiological data like this is literally data that maps information on fibrosis to the geometric topological data of the patient's atrium, it is data that can be generated after the geometric topological data of the patient's atrium is generated, and accordingly, the two data may be regarded as being in a dependent relation, like the anatomical data of the patient's atrium.

Furthermore, since the second electrophysiological data is generated using data that maps voltage information at all points included in the patient's heart, it can be regarded that the data that maps voltage information at all points included in the patient's heart and the second electrophysiological data are also in a dependent relation.

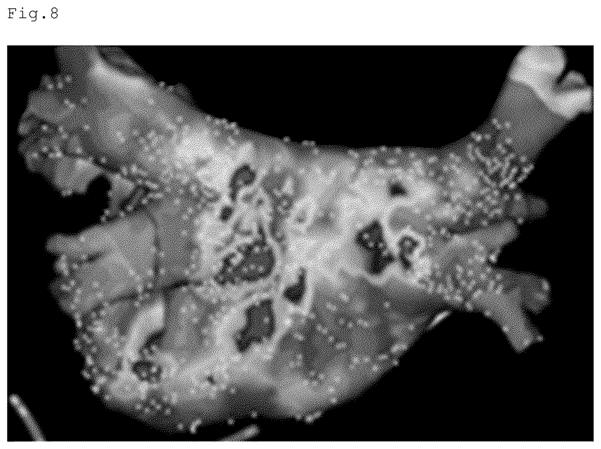

Meanwhile, since the process of mapping voltage information at all points included in the patient's atrium may adopt a method of generating (mapping) a heart voltage map based on a patient's heart model described in Patent Registration No. 10-1964918B1 (Mar. 27, 2019) as it is or partially adopt and apply a method of generating (mapping) a three-dimensional voltage difference atrial model described in Patent Publication No. 10-2020-0095967A (Aug. 11, 2020), detailed description thereof will be omitted to avoid duplicate description, and data that maps the voltage information at all points included in the patient's atrium is exemplarily shown in FIG. 8.

Since the process of generating the second electrophysiological data using data that maps voltage information at all points included in the patient's atrium may also adopt a method of determining the probability of designating a block according to voltage (meaning the same as the probability of fibrosis) and whether or not a block is designated (meaning the same as designation of fibrosis) described in Patent Registration No. 10-1964918B1 (Mar. 27, 2019), and the second electrophysiological data may be easily generated through an operation, detailed description thereof will be omitted to avoid duplicate description.

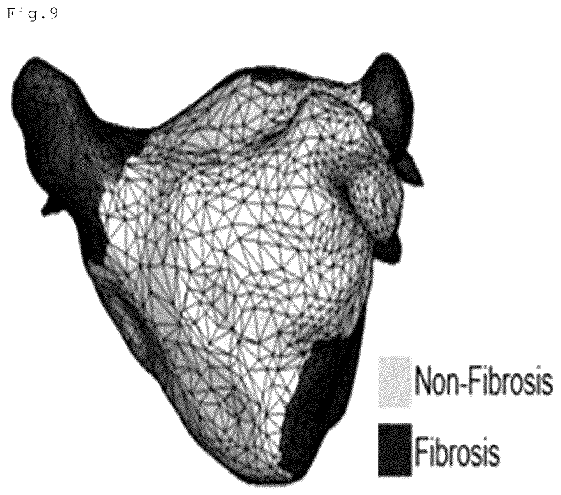

The second electrophysiological data included in the electrophysiological data of a patient's atrium is exemplarily shown in FIG. 9, and since it is data that maps information on fibrosis to a three-dimensional polygon mesh, which is the geometric topological data of the patient's atrium, it can be confirmed that it includes the geometric topological data shown in FIG. 5, more specifically, a plurality of vertices and edges, and a plurality of triangular faces configured of the vertices and edges, and since it can be confirmed that two colors are shown on the surface of the atrium, each color represents fibrosis at all points included in the patient's atrium, and in this case, it can be said that information on fibrosis, as well as location information (x, y, z) of the Euclidean coordinate system, is mapped to each vertex included in the three-dimensional polygon mesh.

So far, target data has been described in detail with reference to FIGS. 3 to 9, and since the present invention predicts recurrence of atrial fibrillation, it has been described by limiting the target data to the patient's atrium, but it will be apparent that the target data may be generated for the entire heart including the patient's ventricles. Now, go back to description of FIG. 2.

When target data is generated, the apparatus 100 including a processor and a memory performs preprocessing on the generated target data (S220).

Preprocessing on the target data performed at step S220 means a process of refining the target data in a form suitable for being input into a prediction model at step S230 described below. This is to prevent the bias that occurs as the number of vertices is different from the number of edges in the three-dimensional polygon mesh acquired for each patient according to features of the patient, and describing briefly, mesh quantification may be regarded as preprocessing. This will be described below.

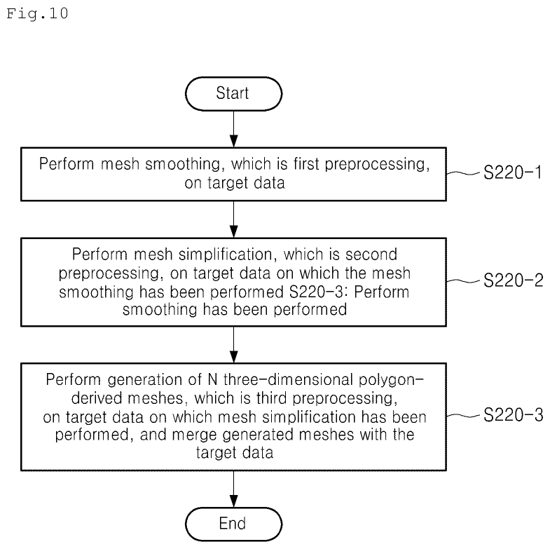

FIG. 10 is a flowchart specifically illustrating step S220 that performs preprocessing on target data in a method of predicting recurrence of atrial fibrillation according to a second embodiment of the present invention.

This is only a preferred embodiment for achieving the objects of the present invention, and some steps may be added or deleted as needed, and furthermore, any one step may be included in another step.

First, mesh smoothing, which is first preprocessing, is performed on the target data (S220-1).

Here, the target data is a concept that includes all of the geometric topological data of the three-dimensional polygon mesh structure of the patient's atrium, the anatomical data to which information on atrial wall thickness is mapped, the first electrophysiological data to which information on local activation time is mapped, and the second electrophysiological data to which information on the presence or absence of fibrosis is mapped as described above, and in some cases, it may even include data to which voltage information is mapped at all points included in the patient's atrium, and therefore, since all of these are generated from a computed tomography image, the rough surface may be changed to be smooth by performing mesh smoothing.

More specifically, the mesh smoothing may be performed by applying the Laplacian Smoothing algorithm, and the number of iterations for the mesh smoothing may be at least three times, and of course, more than three times is possible.

Next, mesh simplification, which is second preprocessing, is performed on the target data on which the mesh smoothing has been performed (S220-2).

Here, the mesh simplification, which is the second preprocessing, may include a step (S220-2-1) of selecting uniform vertices among a plurality of vertices by applying a Poisson disk sampling algorithm, and a step (S220-2-2) of simplifying the selected uniform vertices by applying an edge collapse algorithm. Simplification may be performed to the targeting edge level through step S220-2-2.

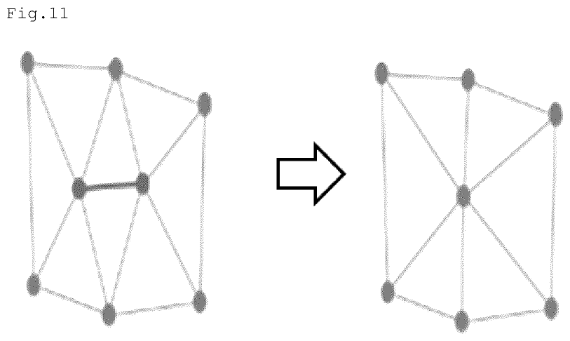

A view of edge simplification according to application of an edge collapse algorithm is exemplarily shown in FIG. 11, and progressive simplification is adopted at step S220-2-2 so that the mesh may be configured of uniform vertices based on the selected uniform vertices while preserving the mesh surface by applying a re-tiling algorithm, and since it is performed under the control condition in which the reference geometric shape (Hausdorff distance) is less than 2 and the structural similarity (SSIM) is less than 0.7, loss in the geometric shape and data due to excessive simplification can be prevented.

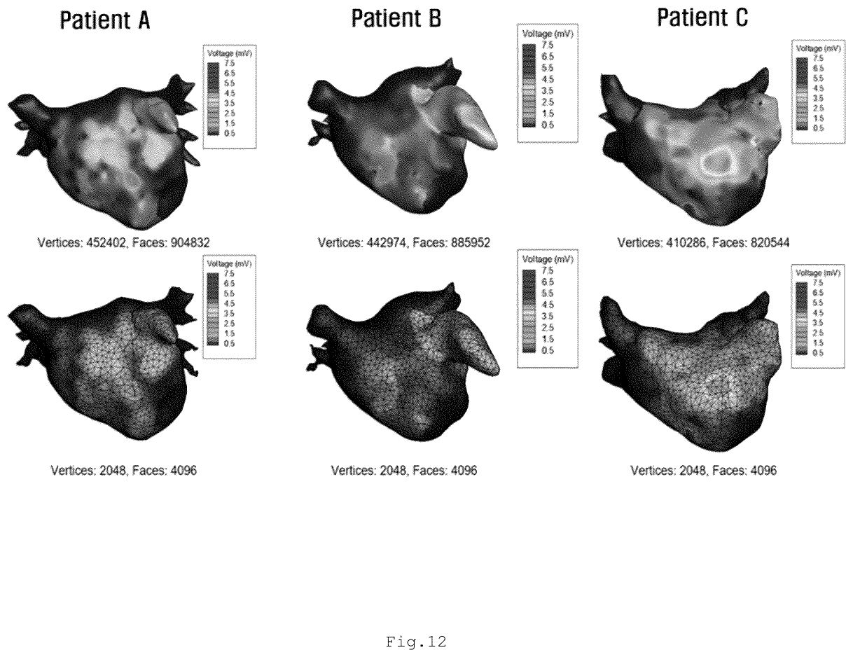

FIG. 12 is a view exemplarily showing the results of performing the method of predicting recurrence of atrial fibrillation as far as mesh simplification, which is the second preprocessing of step S220-2, on the data that maps voltage information at all points included in in the patient's atrium of three patients A, B, and C, and although the number of vertices is different from the number of triangular faces in each patient, since the three-dimensional polygon mesh includes 2,048 vertices and 4,096 triangular faces, it can be confirmed that it is the same without a difference between the patients, and accordingly, the number of edges is 6,144 (2,048*3) to be the same. Although steps S220-1 and S220-2 are not shown separately since they are performed on all target data, it can be said that the numbers of vertices, edges, and triangular faces are the same as well in the case of other target data.

When the method of predicting recurrence of atrial fibrillation is performed as far as the mesh simplification, which is the second preprocessing, generation of N three-dimensional polygon-derived meshes (N is a positive integer), which is third preprocessing, is performed on the target data on which the mesh simplification has been performed, and the generated meshes are merged with the target data (S220-3).

Since the purpose of preprocessing the target data at step S220 is to prevent a bias due to the features of the patient, and prevent learning toward a biased result according to various topologies that may be generated although the shapes are the same since the target data adopts a three-dimensional polygon mesh as the basic structure, step S220-3 corresponds to a step for this purpose.

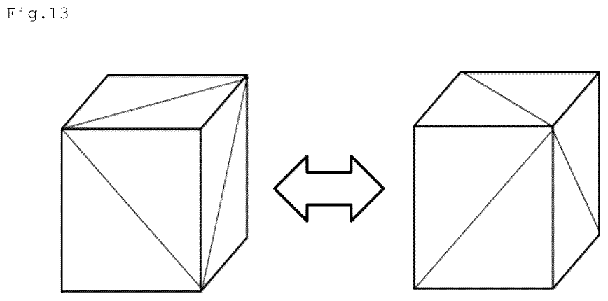

FIG. 13 is a view exemplarily showing a case where topologies are different although the shapes are the same. Although both the left and right sides include seven vertices and three edges, it can be confirmed that different topologies are shown as the edges connecting the vertices are different.

Meanwhile, when N is too small in performing generation of a three-dimensional polygon-derived mesh, there is a problem in that various topologies may not be reflected sufficiently, and when N is too large, there is a problem in that an excessively long time is required for calculation and the apparatus 100 including a processor and a memory may be overloaded although various topologies may be sufficiently reflected. Therefore, various topologies may be effectively reflected and efficiency in calculation may be improved by setting N to 5, and it goes without saying that this can be changed without limit according to the setting of a user or a manager of the apparatus 100 including a processor and a memory.

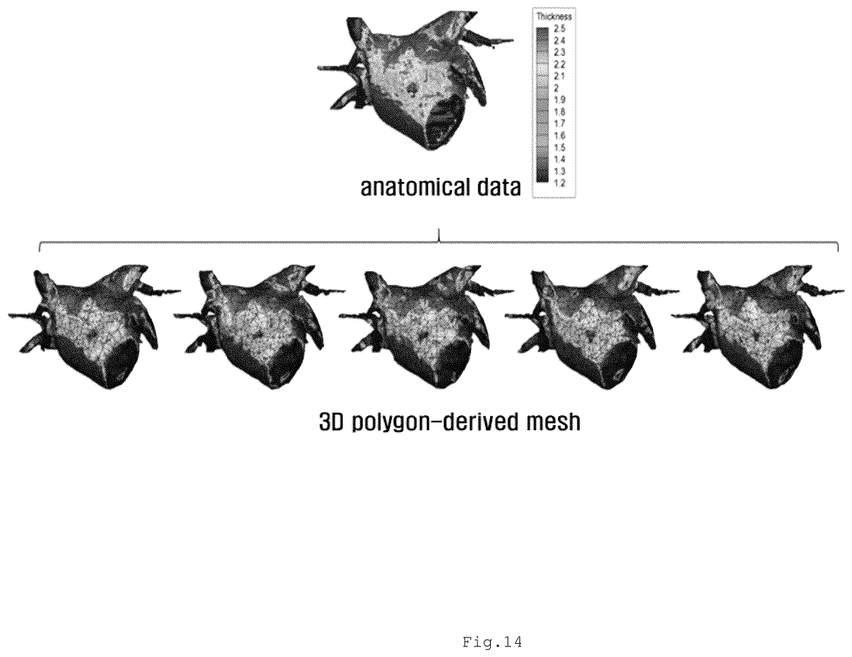

FIG. 14 exemplarily shows a view of generating five three-dimensional polygon-derived meshes by setting N to 5 for the anatomical data of the patient's atrium, which is the target data, more specifically for the data that maps information on atrial wall thickness at all points included in the patient's atrium expressed three-dimensional polygon mesh, which is the geometric topological data of the patient's atrium. It can be confirmed that although information on atrial wall thickness mapped to each point is the same, edges connecting vertices are different.

Meanwhile, the merge at step S220-3 should be interpreted as adding the generated three-dimensional polygon-derived mesh to the target data, rather than merging the contents of actual data.

Now, go back to description of FIG. 2.

When the method of predicting recurrence of atrial fibrillation is performed as far as preprocessing on the target data, the apparatus 100 including a processor and a memory inputs the preprocessed target data into a prediction model to calculate and output a prediction value of recurrence of atrial fibrillation of the patient, and learns the prediction value (S230).

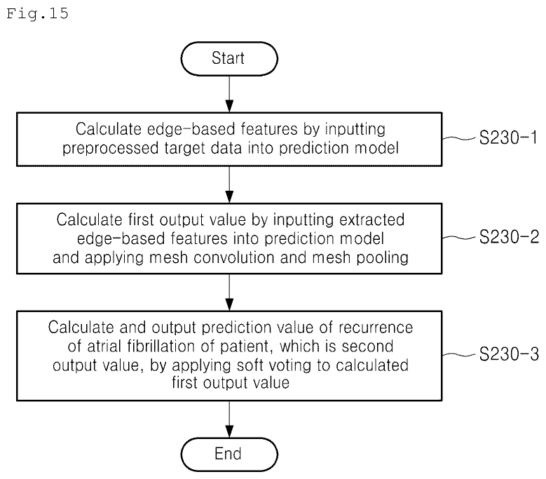

Here, since the three-dimensional polygon mesh has an unstructured data structure, the prediction model may not calculate and output a prediction value using a prediction model that processes a fixed grid form like a conventional image, a unique prediction model optimized for the present invention should be implemented, and accordingly, step S230 may be subdivided as follows. This will be described below with reference to FIG. 15.

FIG. 15 is a flowchart specifically illustrating step S230 of calculating and outputting a prediction value of recurrence of atrial fibrillation in a method of predicting recurrence of atrial fibrillation according to a second embodiment of the present invention.

This is only a preferred embodiment for achieving the objects of the present invention, and some steps may be added or deleted as needed, and furthermore, any one step may be included in another step.

First, edge-based features are calculated by inputting the preprocessed target data into the prediction model (S230-1).

Here, as the edge-based features mean geometric features, which are diameters at all edges, and mapping information at all edges, the calculated edge-based features may vary according to the type of target data.

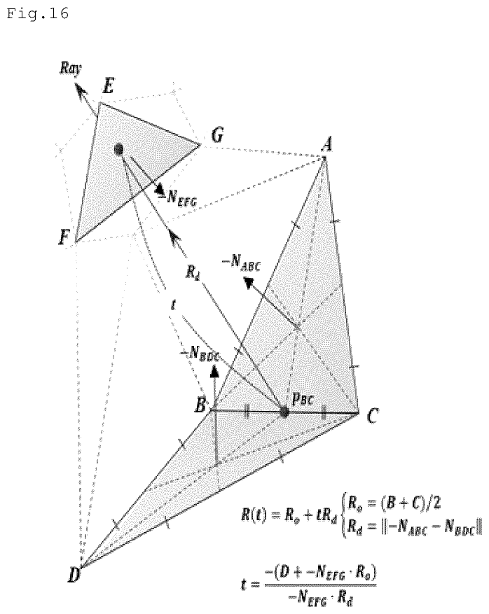

More specifically, when the target data includes geometric topological data, i.e., when the target data is geometric topological data, step S230-1 may include a step of inputting the preprocessed target data into a prediction model and calculating a diameter at all edges, which is a first edge-based feature (S230-1-1), and here, the diameter at all edges may be calculated by estimating the distance (a sphere) from the center point of each edge to the point where a ray penetrates the interior of the three-dimensional object and collides with the inner surface of the opposite side, through a ray casting method as exemplarily shown in FIG. 16.

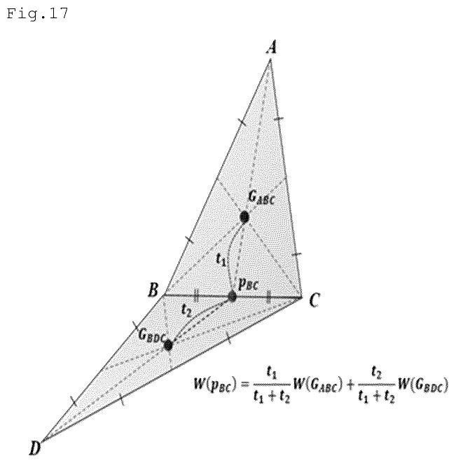

Meanwhile, when the target data includes any one or more among anatomical data and electrophysiological data, i.e., when the target data is anatomical data or electrophysiological data, step S230-1 may include a step of inputting the preprocessed target data into a prediction model and calculating mapping information at all edges, which is a second edge-based feature (S230-1-2), and as exemplarily shown in FIG. 17, the mapping information at all edges may be calculated by estimating the center of gravity of two triangles including any one edge for which mapping information is to be calculated, estimating the distance from the center of gravity to the center point of any one edge, and applying a linear interpolation method.

Step S230-1, which is the characteristic of the prediction model related to the edge-based feature extraction described above, has an advantage in that invariance to the transformation of movement and rotation generated by the movement and posture of a patient while performing computed tomography can be guaranteed as the mapping information is estimated relatively based on the edges, and the computing speed can be dramatically improved as only the diameter is calculated compared to the conventional mesh-based prediction model that calculates dihedral angles, interior angle of the vertices, a relative edge ratio, and the like.

Thereafter, a first output value is calculated by inputting the extracted edge-based features into the prediction model and applying mesh convolution and mesh pooling (S230-2).

Here, since the mesh convolution and the mesh pooling are performed by applying the convolution operators and edge collapse algorithm disclosed in the well-known paper Mesh CNN (Hanocka et al.), detailed description thereof will be omitted to avoid duplicate description.

Finally, a prediction value of recurrence of atrial fibrillation of the patient, which is the second output value, is calculated and output by applying soft voting to the calculated first output value (S230-3).

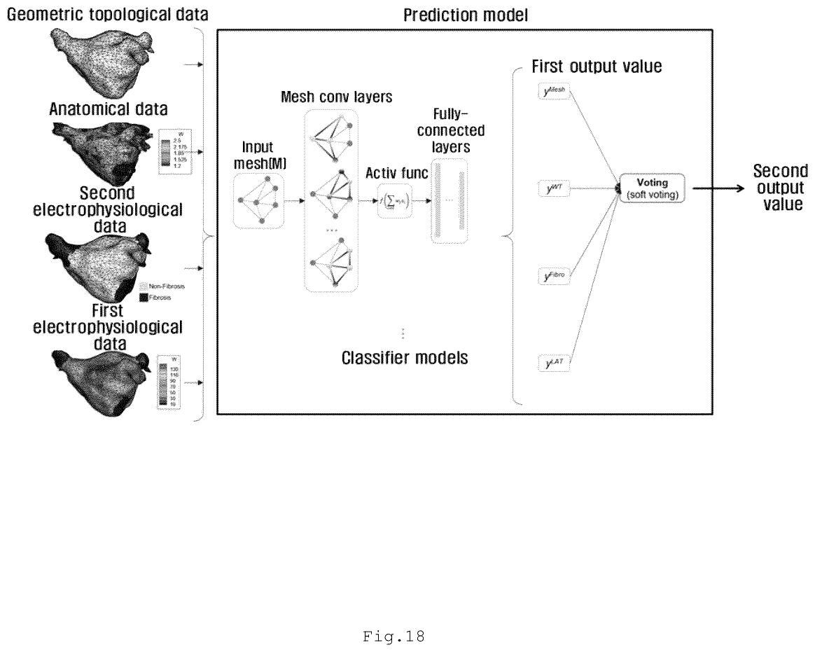

A view of calculating a first output value by inputting target data into the prediction model, and calculating and outputting a prediction value of recurrence of atrial fibrillation of the patient, which is the second output value, by applying soft voting to the first output value is shown in FIG. 18, and since soft voting is applied, the probabilities of determining label values of classifiers are all added up and averaged, and a label value with the highest probability is calculated and output as the final second output value.

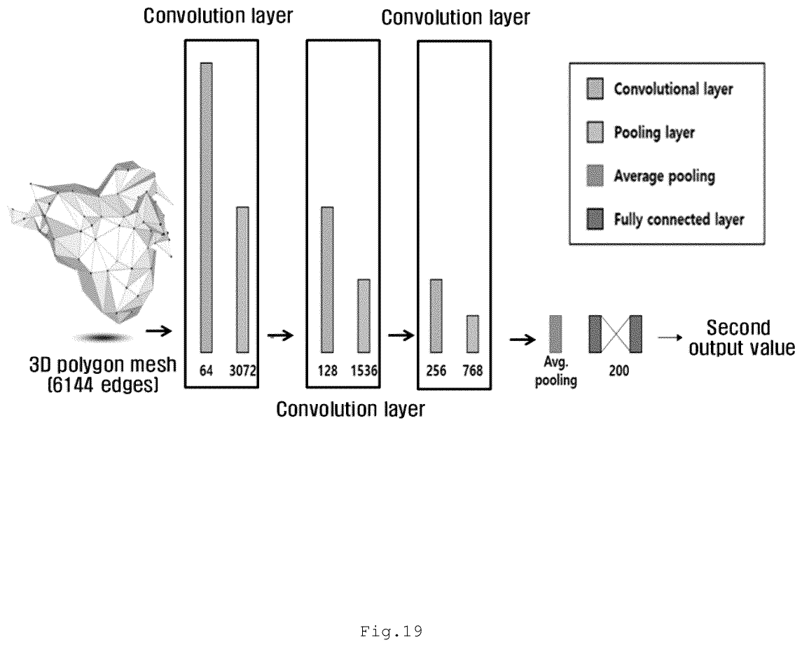

According to the explanation described above, the prediction model is a model configured as an ensemble-based method that uses all of geometric topological data, anatomical data, and electrophysiological data, which are target data, and it is since that it is better from the aspect of prediction accuracy to perform prediction by comprehensively applying heterogeneous data rather than explaining the predictive power with data of single objects since atrial fibrillation is a geriatric disease that accompanies various comorbidities in many cases. FIG. 19 exemplarily shows a single mesh neural network constituting a prediction model, which is largely configured of three convolutional layers, and it can be confirmed that the initial input of 6,144 edges is reduced to 3,072, 1,536, and 768 edges as it passes through each convolutional layer by performing edge collapse of 0.5 times thereon, and it can be confirmed that 64, 128, and 256 convolution kernels are configured in each layer.

The method of predicting recurrence of atrial fibrillation according to a second embodiment of the present invention has been described so far. According to the present invention, recurrence of atrial fibrillation can be predicted rapidly with high accuracy using only computed tomography images, information on atrial wall thickness, information on local activation time, and information on fibrosis, which are the minimum level of information on the patient's heart. In addition, since the information described above is information that is generally possessed by a patient of atrial fibrillation and is also information that the apparatus 100 including a processor and a memory may definitively generate through an operation, a new invasive test, which is burdensome to the patient's body, does not need to be performed to acquire new information, and accordingly, physical and economic burdens of the patient can be minimized. In addition, since a prediction value of recurrence of atrial fibrillation is calculated and automatically output by simply generating and inputting target data into a prediction model, and the prediction value is continuously learned, accuracy of predicting recurrence of atrial fibrillation can be improved as the present invention is repeatedly used.

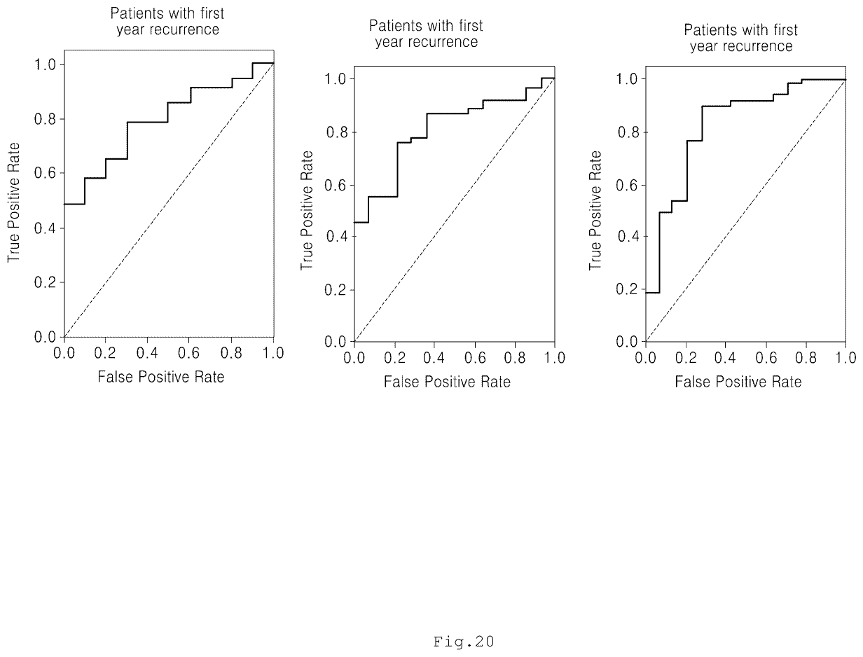

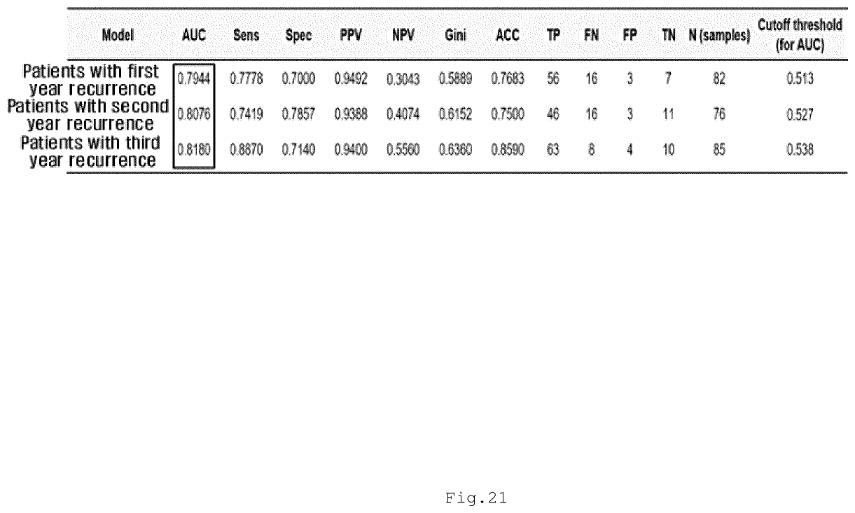

FIG. 20 and FIG. 21 show ROC curves and a table showing the results of long-term observation of recurrence of atrial fibrillation by year recorded in the EMR based on data of patients with recurrence of atrial fibrillation after actual radiofrequency catheter ablation, in a method of predicting recurrence of atrial fibrillation according to a second embodiment of the present invention, and it can be confirmed that the predictive power for the patients with long-term recurrence is reliable since the AUC of the 1st year recurrence to the 5th year recurrence is 0.79 to 0.82.

Meanwhile, although it has not been described in detail to avoid duplicate description, the apparatus 100 for predicting recurrence of atrial fibrillation according to a first embodiment of the present invention and the method of predicting recurrence of atrial fibrillation according to a second embodiment of the present invention may be implemented as a computer program stored in a medium according to a third embodiment of the present invention including the same technical features. In this case, the computer program stored in the medium is combined with a computing device and includes the steps of (AA) generating target data including any one or more among geometric topological data, anatomical data, and electrophysiological data of a patient's atrium, (BB) performing preprocessing on the generated target data, and (CC) inputting the preprocessed target data into a prediction model, and calculating and outputting a prediction value of recurrence of atrial fibrillation of the patient, and the target data is defined by a plurality of vertices and edges, and adopts a three-dimensional polygon mesh including a plurality of triangular faces, i.e., minimum units of faces configured of vertices and edges, as a basic structure, and the computer program may derive an effect the same as that of the apparatus 100 for predicting recurrence of atrial fibrillation according to a first embodiment of the present invention and the method of predicting recurrence of atrial fibrillation according to a second embodiment of the present invention.

Although the embodiments of the present invention have been described above with reference to the accompanying drawings, those skilled in the art may understand that the present invention can be implemented in other specific forms without changing the technical spirit or essential features. Therefore, the embodiments described above should be understood as illustrative and not restrictive in all respects.

1. A method of predicting recurrence of atrial fibrillation through an apparatus including a processor and a memory, the method comprising the steps of:
(a) generating target data including any one or more among geometric topological data, anatomical data, and electrophysiological data of a patient's atrium;
(b) performing preprocessing on the generated target data; and
(c) inputting the preprocessed target data into a prediction model, calculating and outputting a prediction value of recurrence of atrial fibrillation of the patient, and learning the prediction value, wherein
the target data is defined by a plurality of vertices and edges and adopts a three-dimensional polygon mesh including a plurality of triangular faces, i.e., minimum units of faces configured of vertices and edges, as a basic structure.

2. The method according to claim 1, wherein the geometric topological data of the patient's atrium is data generated as a three-dimensional polygon mesh structure by applying a marching cubes algorithm while structures adjacent to the heart are excluded from a computed tomography (CT) image of the patient's heart and only segmented shapes of the atrium are extracted.

3. The method according to claim 2, wherein the anatomical data of the patient's atrium is data that maps information on atrial wall thickness at all points included in the patient's atrium expressed as a three-dimensional polygon mesh, which is the geometric topological data of the patient's atrium.

4. The method according to claim 2, wherein the electrophysiological data of the patient's atrium includes first electrophysiological data that maps information on local activation time at all points included in the patient's atrium expressed as a three-dimensional polygon mesh, which is the geometric topological data of the patient's atrium.

5. The method according to claim 2, wherein the electrophysiological data of the patient's atrium is generated using data that maps voltage information at all points included in the patient's atrium expressed as a three-dimensional polygon mesh, which is the geometric topological data of the patient's atrium, as well as the first electrophysiological data, and includes second electrophysiological data that maps information on fibrosis at all points included in the patient's atrium expressed as a three-dimensional polygon mesh, which is the geometric topological data of the patient's atrium.

6. The method according to claim 1, wherein step (b) includes the steps of:
(b-1) performing mesh smoothing, which is first preprocessing, on the generated target data;
(b-2) performing mesh simplification, which is second preprocessing, on the target data on which the mesh smoothing has been performed; and
(b-3) performing generation of N three-dimensional polygon-derived meshes (N is a positive integer), which is third preprocessing, on the target data on which the mesh simplification has been performed, and merging the generated mesh with the target data.

7. The method according to claim 6, wherein the mesh smoothing, which is first preprocessing performed at step (b-1), is performed by applying a Laplacian smoothing algorithm, and an iterative operation for the Laplacian Smoothing is performed three times.

8. The method according to claim 6, wherein the mesh simplification, which is second preprocessing, performed at step (b-2) includes the steps of:
(b-2-1) selecting uniform vertices among the plurality of vertices by applying a Poisson disk sampling algorithm; and
(b-2-2) simplifying the selected uniform vertices by applying an edge collapse algorithm, wherein
at step (b-2-2), the mesh is configured of uniform vertices based on the selected uniform vertices while preserving a mesh surface by applying a re-tiling algorithm.

9. The method according to claim 6, wherein the mesh simplification, which is second preprocessing, performed at step (b-2) is performed under a control condition in which a reference geometric shape (Hausdorff distance) is less than 2 and a structural similarity (SSIM) is less than 0.7.

10. The method according to claim 6, wherein the target data on which the method has been performed as far as step (b-2) is a three-dimensional polygon mesh structure including 2,048 vertices, 6,144 edges, and 4,096 faces.

11. The method according to claim 6, wherein in generation of derived meshes at step (b-3), which is third preprocessing, N is 5, and the three-dimensional polygon-derived meshes are three-dimensional polygon meshes having a shape the same as that of the target data on which the mesh simplification has been performed, and are meshes having edges to which the vertices are connected are different.

12. The method according to claim 1, wherein step (c) includes the steps of:
(c-1) calculating edge-based features by inputting the preprocessed target data into the prediction model;
(c-2) calculating a first output value by inputting the extracted edge-based features into the prediction model and applying mesh convolution and mesh pooling; and
(c-3) calculating and outputting the prediction value of recurrence of atrial fibrillation of the patient, which is a second output value, by applying soft voting to the calculated first output value.

13. The method according to claim 12, wherein when the target data includes geometric topological data, step (c-1) includes (c-1-1) a step of inputting the preprocessed target data into the prediction model and calculating a diameter at all edges, which is a first edge-based feature, wherein the diameter at all the edges is calculated by estimating collision points from each edge to an opposite surface through ray casting.

14. The method according to claim 12, wherein when the target data includes any one or more among anatomical data and electrophysiological data, step (c-1) includes (c-1-2) a step of inputting the preprocessed target data into the prediction model and calculating mapping information at all edges, which is a second edge-based feature, wherein the mapping information at all edges is calculated by estimating a center of gravity of two triangles including any one edge for which mapping information is to be calculated, estimating a distance from the center of gravity to a center point of any one edge, and applying a linear interpolation method.

15. An apparatus for predicting recurrence of atrial fibrillation, the apparatus comprising:
one or more processors;
a network interface;
a memory that loads a computer program executed by the processor; and
a storage that stores a large amount of network data and the computer program, wherein
the computer program executes, by the one or more processors,
(A) an operation of generating target data including any one or more among geometric topological data, anatomical data, and electrophysiological data of a patient's atrium;
(B) an operation of performing preprocessing on the generated target data; and
(C) an operation of inputting the preprocessed target data into a prediction model, calculating and outputting a prediction value of recurrence of atrial fibrillation of the patient, and learning the prediction value, wherein
the target data is defined by a plurality of vertices and edges and adopts a three-dimensional polygon mesh including a plurality of triangular faces, i.e., minimum units of faces configured of vertices and edges, as a basic structure.

16. A computer program stored in a medium, the program comprising, in combination with a computing device, the steps of:
(AA) generating target data including any one or more among geometric topological data, anatomical data, and electrophysiological data of a patient's atrium;
(BB) performing preprocessing on the generated target data; and
(CC) inputting the preprocessed target data into a prediction model, calculating and outputting a prediction value of recurrence of atrial fibrillation of the patient, and learning the prediction value, wherein
the target data is defined by a plurality of vertices and edges and adopts a three-dimensional polygon mesh including a plurality of triangular faces, i.e., minimum units of faces configured of vertices and edges, as a basic structure.
